(19) United States
(12) Patent Application Publication
(10) Pub. No.: US 2017/0325949 A1
(43) Pub. Date: Nov. 16, 2017

(54) Heart Valve Implant And Methods For Delivering And Implanting Same

(71) Applicant: Cardiosolutions, Inc., Bridgewater, MA (US)

(72) Inventors: Richard Rodgers, Hudson, MA (US); Steve Tallarida, Mansfield, MA (US)

(21) Appl. No.: 15/591,780

(22) Filed: May 10, 2017

Related U.S. Application Data
(60) Provisional application No. 62/336,210, filed on May 13, 2016.

(51) Int. Cl. A61F 2/24; A61L 27/50; A61L 27/14; A61L 27/06

(52) U.S. Cl. CPC A61F 2/2421; A61F 2/2436; A61F 2/2457; A61F 2/2487; A61L 27/14; A61L 27/06; A61F 2/2403; A61L 27/50; A61F 2210/0014; A61L 2400/16; A61L 2430/20; A61F 2220/0016

(57) ABSTRACT

Heart valve implants and methods for implanting and delivering same are described. A heart valve implant can include a shaft, having a first end and a second end, an anchor, and a plurality of wafers. The anchor is coupled to the first end of the shaft and configured to secure the heart implant to a patient's heart. The wafers are coupled to the second end of the shaft and configured to form a stacked array of wafers. The stacked array of wafers can partially reduce a flow of blood through a heart valve upon coming in contact with a portion of a leaflet of the heart valve.

### RELATED APPLICATIONS

This application claims the benefit of and priority to U.S. Provisional Application 62/336,210, filed on May 13, 2016, the entire teachings of which are incorporated by reference herein.

### TECHNICAL FIELD

The present disclosure relates generally to the repair and/or correction of dysfunctional heart valves, and more particularly to heart valve implants, systems, and methods for delivery and implantation of such implants.

### BACKGROUND

A human heart has four chambers, including the left and right atriums and the left and right ventricles. The chambers of the heart alternately expand and contract to pump blood through the vessels of the body. The cycle of the heart includes the simultaneous contraction of the left and right atria, passing blood from the atria to the left and right ventricles. The left and right ventricles then simultaneously contract, forcing blood from the heart and through the vessels of the body. In addition to the four chambers, the heart also includes a check valve at the upstream end of each chamber to ensure that blood flows in the correct direction through the body as the heart chambers expand and contract. These valves can become damaged or otherwise fail to function properly, resulting in their inability to properly close when the downstream chamber contracts. Failure of the valves to properly close can allow blood to flow backward through the valve, resulting in decreased blood flow and lower blood pressure.

Mitral regurgitation is a common variety of heart valve dysfunction or insufficiency. Mitral regurgitation occurs when the mitral valve separating the left coronary atrium and the left ventricle fails to close properly. As a result, upon contraction of the left ventricle, blood can leak or flow from the left ventricle back into the left atrium, rather than being forced through the aorta. Any disorder that weakens or damages the mitral valve can prevent the mitral valve from closing properly, thereby causing leakage or regurgitation. Mitral regurgitation is considered to be chronic when the condition persists rather than occurring for only a short period of time.

Regardless of the cause, mitral regurgitation can result in a decrease in blood flow through the body (e.g., reduce cardiac output). Correction of mitral regurgitation typically requires surgical intervention. Surgical valve repair or replacement can be carried out as an open-heart procedure. The repair or replacement surgery can last in the range of about three to five hours, and can be carried out with the patient under general anesthesia. The nature of the surgical procedure requires the patient to be placed on a heart-lung machine. Because of the severity, complexity, and/or danger associated with open-heart surgical procedures, corrective surgery for mitral regurgitation cannot be recommended in certain patients.

### SUMMARY

Heart valve devices and methods of implanting the same are described herein. In one aspect, a heart valve implant is described. The heart valve can comprise a shaft, having a first end and a second end, an anchor, coupled to the first end of the shaft and configured to secure the heart valve implant to a patient's heart, and a plurality of wafers, wherein each of the plurality of wafers is coupled to the second end of the shaft to form a stacked array of wafers.

In another aspect, a method for delivering or implanting a heart valve implant is described. The described method comprises: percutaneously inserting the heart valve implant in a patient's heart, wherein the heart valve implant comprises a shaft having a first end and a second end, an anchor coupled to said first end of said shaft, the anchor configured to secure the heart valve implant; and a plurality of wafers, wherein each of the plurality of wafers is coupled to the second end of the shaft. The implanting step comprises at least partially collapsing the plurality of wafers of said heart valve implant, percutaneously inserting said heart valve implant into the heart, securing said anchor of said heart valve implant to native coronary tissue of the said heart (e.g., within the native coronary tissue, within the left ventricle, etc.); and expanding the plurality of wafers to form a stacked array of wafers at least partially within a heart valve to at least partially restrict a flow of blood through a heart valve during systole upon contact with at least a portion of a valve leaflet of the heart valve.

In yet another aspect, a method for delivering a heart valve implant is described. The described method includes percutaneously inserting a heart valve implant into a patient's heart, wherein the heart valve implant comprises a shaft extending from a first end to a second end, an anchor coupled to the first end of the shaft, and a plurality of wafers coupled to the shaft in proximity of the second end of the shaft, and wherein said wafers are in an at least partially collapsed state. The described method further includes securing said anchor of the heart valve implant to native coronary tissue and expanding the plurality of wafers to form a stacked array of wafers disposed at least partially within the heart valve so as to at least partially restrict a flow of blood through the heart valve during systole.

In other examples, any of the aspects above, or any system, method, apparatus described herein can include one or more of the following features.

The stacked array of wafers can be configured to at least partially restrict a flow of blood through the heart valve during systole upon contact with at least a portion of a valve leaflet of the heart valve. The stacked array of wafers can comprise a compliant surface.

The heart valve implant can further comprise an inflatable balloon. The balloon can surround at least a portion of the plurality of wafers. The balloon can be at least partially inflated with a fluid.

In some embodiments, the plurality of wafers, by way of example, can comprise a biocompatible polymer, a shape memory material, or a combination thereof. The biocompatible polymer can comprise polyurethane, a polyethylene terephthalate (PET) and polyethyleneoxide (PEO) block copolymer, a polystyrene and poly(1,4-butadiene) block copolymer, a triblock copolymer made from poly(2-methyl-2-oxazoline) and polytetrahydrofuran, a polysiloxane, a polyether, or a combination thereof. In some embodiments, the shape memory material can comprise nitinol.

The stacked array can comprise a distal end and a proximal end. The stacked array of wafers can exhibit a tapered shape extending from a proximal end to a distal end. The stacked array of wafers can exhibit a diamond-like shape or cross-section. The cross-sectional shape of the stacked array of wafers can include at least one of round, pear-shaped, elliptical, hour-glass shaped, triangular and heart shaped.

In some embodiments, the plurality of wafers can have substantially equal cross-sectional diameters. The cross-sectional shape of the stacked array of wafers can include at least one of rectangle or square shape.

The plurality of wafers can be slidably coupled to the shaft. Alternatively or additionally, the plurality of wafers can be fixedly coupled to the shaft. The heart valve implant can further comprise a plurality of coupling members. Each of the plurality of coupling members can be configured to couple at least one of the wafers to the shaft.

The plurality of wafers can be surrounded all or in part with an outer body or covering (such as a balloon). Such covering can be connected to or separate from the wafers. Such a balloon can be partly filled with fluid.

Each of the plurality of wafers can include holes, cut-out sections, and/or hollow areas within their body. The wafers can be interconnected at their attachment to the shaft or at locations along the surfaces between the wafers. Such interconnections can comprise one or more materials used in the body of the wafers or of other materials.

The heart valve implant can comprise one or more radiopaque markers. The one or more radiopaque markers can be disposed on the shaft, one or more of the plurality of wafers, or a combination thereof.

The anchor of the heart valve implant can comprise at least one barb, a helical feature, at least one anchor pad, or a combination thereof.

In some embodiments, the heart valve implant can be percutaneously inserted using a catheter. For example, the plurality of wafers can be collapsed and inserted into a lumen of the catheter and the heart valve implant can be delivered to a ventricle (e.g., left ventricle) via said catheter.

The heart valve implant can be secured to any of an interior surface of the heart, an exterior surface of the heart, or a combination thereof. In some embodiments, the heart valve implant can be a mitral valve implant.

Other aspects and advantages of the invention can become apparent from the following drawings and description, all of which illustrate the principles of the invention, by way of example only.

### BRIEF DESCRIPTION OF THE DRAWINGS

Features and advantages of the invention described herein, together with further advantages, may be better understood by referring to the following description taken in conjunction with the accompanying drawings. The drawings are not necessarily to scale, emphasis instead is generally placed upon illustrating the principles of the invention

FIG. 1 is a perspective view of an embodiment of a heart valve implant according to some embodiments disclosed herein.

FIG. 2 depicts an embodiment of a mitral valve implant consistent with the present disclosure implanted within a heart in a closed position.

FIG. 3 depicts an embodiment of a mitral valve implant implanted within a heart.

FIGS. 4A-4B are cross-sectional views of other embodiments having a plurality of wafers consistent with the present disclosure.

FIGS. 5A, 5B, and 5C are cross-sectional views of other embodiments having a plurality of wafers consistent with the present disclosure.

FIG. 6A is a perspective view of an embodiment of a heart valve implant.

FIG. 6B is a top view of the heart valve implant of FIG. 6A.

FIGS. 7A-7D are cross-sectional views of other embodiments of implants having a plurality of wafers consistent with the present disclosure.

FIG. 8 is a perspective view of an embodiment of a mitral valve implant shown in an expanded position.

FIGS. 9A-9B are cross-sectional views of an embodiment of a mitral valve implant that includes a balloon surrounding the wafers.

FIG. 10A is a cross-sectional view of another embodiment of a heart valve implant having a balloon that surrounds a plurality of wafers consistent with the present disclosure.

FIG. 10B illustrates a heart valve implant having one or more radiopaque markers.

### DETAILED DESCRIPTION

Heart implants and methods and systems for implanting and delivering the implants are described. The heart valve implants described herein can be used in connection with the treatment and/or correction of a dysfunctional or inoperative heart valves. For example, the heart valve implant described herein can be used for treatment and/or correction of mitral valve regurgitation.

Although implementations of the embodiments described herein can be used for correction and/or treatment of various heart valve (e.g., mitral, aortic, pulmonary, tricuspid) conditions, diseases, or malfunctions, for the ease of explanation, the heart valve implants herein are described in terms of mitral valve implants and in relation to the treatment of mitral valve regurgitation. However, a heart valve implant according to the embodiments of the present disclosure can be configured for treating, use in, and/or correcting other dysfunctional or inoperative heart valves. The present disclosure should not, therefore, be construed as being limited to use as a mitral valve implant and/or for treatment of mitral valve malfunctions.

Generally, a heart valve implant according to the present disclosure can interact with at least a portion of an existing damaged heart valve to correct the heart valve's dysfunction, e.g., to prevent and/or reduce regurgitation. For example, at least a portion of one or more cusps of the heart valve can interact with, engage, and/or seal against at least a portion of the heart valve implant when the heart valve is in a closed position. By way of example, the interaction, engagement and/or sealing between at least a portion of at least one cusp of a valve (e.g., a mitral valve) and at least a portion of the heart valve implant can reduce and/or eliminate regurgitation in the heart valve. For example, a heart valve may not be able to provide sufficient sealing upon closure due to a variety of different defects, including damage to a single cusp of the valve or removal of a diseased and/or damaged cusp, or a ruptured cordae. A heart valve implant according to the present disclosure can be used in connection with these and/or alternative, or additional, defects and/or deficiencies.

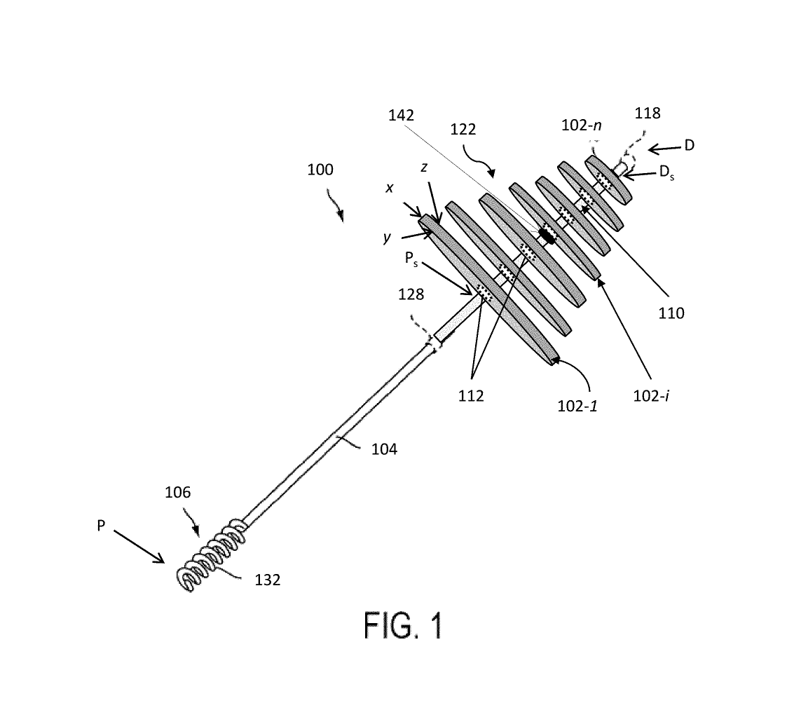

Referring to FIG. 1, a perspective view of an embodiment of a mitral valve implant 100 according to an embodiment of the present teachings is depicted. In general, the mitral valve implant 100 can increase the sealing and/or closure of the passage between the left ventricle and the left atrium during contraction of the left ventricle relative to damaged and/or leaking native valve. Accordingly, in some embodiments the mitral valve implant 100 can be configured to operate in combination with a partially operable and/or damaged mitral valve. That is, the mitral valve implant can interact and/or cooperate with at least a portion of the native mitral valve to reduce and/or eliminate regurgitation.

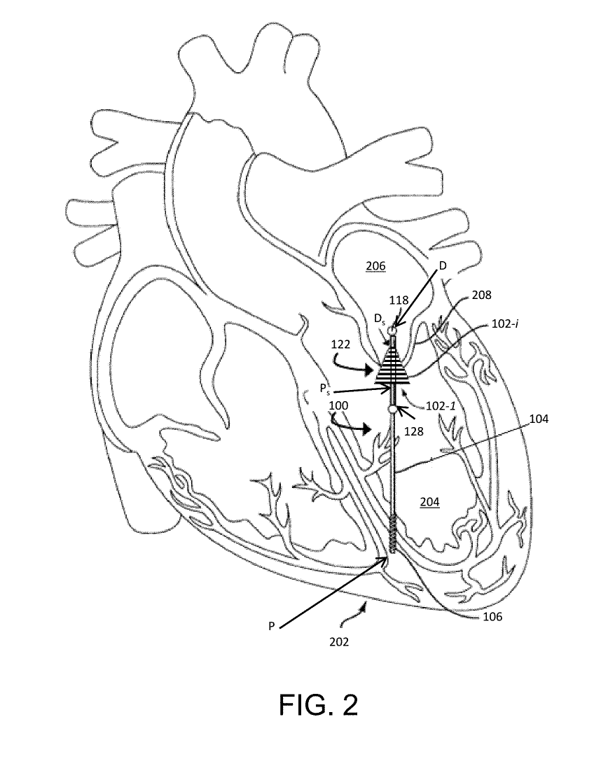

As shown, the mitral valve implant can generally include a plurality of wafers 102-i, where i=1, 2, . . . , n, and nεN. In some embodiments, the number of the wafers can be in average 1 to about 100. The plurality of wafers can form a stacked array of wafers 122, which are coupled to a portion of the second end (e.g., distal end, D) of a shaft 104. The first end (e.g., proximal end, P) of the shaft 104 can be coupled to an anchor portion 106. The anchor portion 106 can be configured to secure the implant to a patient's heart (not shown, see, e.g., FIG. 2).

The implant 100 can further include one or more coupling members 142 that are couple at least one of the wafers 102-i to the shaft 104. For example, as shown in FIG. 1, a coupling member 142 can couple a wafer 102-i to the shaft 104. Each of the wafers 102-i can be fixedly or slidably attached to the shaft 104.

As noted, the wafers 102-i of the mitral valve implant 100 can form a stacked array of wafers 1622. The stacked array of wafers can have a tapered profile. The tapered profile can be characterized by a decrease in the cross-sectional area of the wafers 102-i from a proximal end (Pₛ) to a distal end (Dₛ) of the stacked array 122.

For example, in the embodiment shown in FIG. 1, each wafer 102-i is in the form of a disc circumscribed by a lateral side (x), a top side (z), and a bottom side (y). The cross-sectional area of the disks decreases from a first disk/wafer 102-1, positioned at the proximal end (Pₛ) of the wafer array, to a last disk/wafer 102-n, positioned at the distal end (Dₛ) of the wafer array 122.

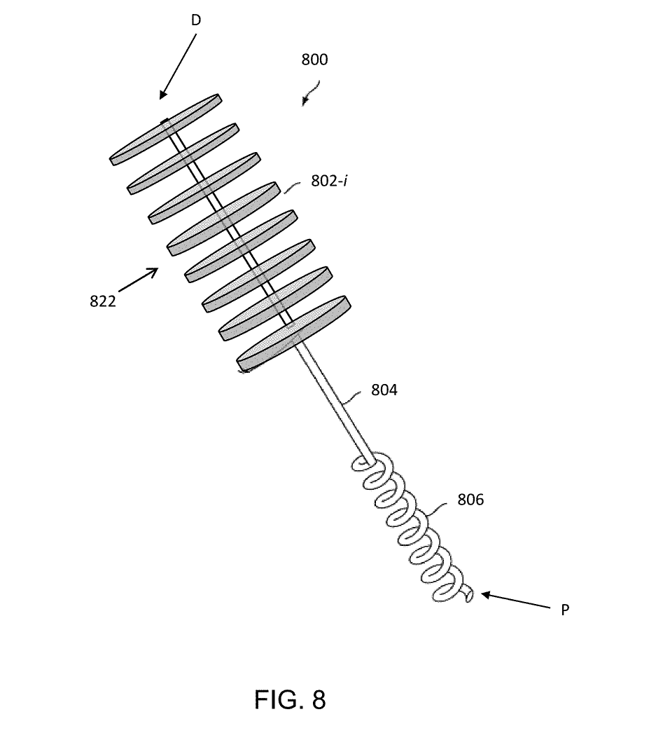

The rate of the change in the cross-sectional areas of the wafers 102 can be linear or non-linear. For example, a non-linear change in the cross-sectional areas of the wafers from the first wafer 102-1 to the last wafer 102-n can result in a taper having a flared or billed shape, leading, e.g., to an at least partially concave taper profile. In some embodiments, the wafers 102-i included in the stacked array of wafers 122 can be substantially uniform in size, and, form a generally straight profile once stacked (see, e.g., FIG. 8). Alternatively and/or additionally, the stacked array of wafers 122 can exhibit a convex profile, producing an at least partially outwardly bulging tapered profile.

In the example illustrated in FIG. 1, the wafers 102-i have a substantially uniform thickness. Further, the axial separations 110, between consecutive wafers, can be substantially uniform. In other implementations, two or more of the wafers can have different thicknesses. Additionally/alternatively, the axial separations 110 between consecutive wafers can be non-uniform. Generally, the spacing 110 between the wafers 102-i and/or the thickness of the wafers 102-i can be uniform or non-uniform.

Further, the surface of each wafer 102-i can be smooth, non-smooth, or a compliant surface (e.g., the wafer has the ability to change upon application of a force or pressure), to allow for non-turbulent flow of blood over each wafer. Additionally and/or alternatively, the wafers can vary in size, causing the stacked array of wafers 122 to have other shapes than the tapered shape depicted in the example shown in FIG. 1.

The wafers 102-i can be slidably coupled to the shaft 104. Each wafer 102-i can include an opening 112 that allows the wafer to be received by and advanced over shaft 1604. In some embodiments, the shaft 104 can extend through opening 1612 of the wafers 102-I to form a stacked array. The opening 112 can be sized to slidably receive at least a portion of the shaft 104 therethrough.

The shaft 104 can include one or more stops 118 or 128. The stops 118 and 128 can be sized and/or shaped to control and/or restrict translation of the wafers 102-i along the shaft 104 beyond the stops 118 and 128. In this manner, as illustrated in FIG. 1, translation of the wafers 102-i along the shaft 104 can be restricted to the expanse of the shaft 104 between the stops 118 and 128. In some other embodiments, the wafers 102-i can be fixedly attached to the shaft 104.

The stops 118, 128 can be integrally formed with the shaft 104. Alternatively, the stops 118, 128 can be provided as a separate member and coupled to the shaft 104. In embodiments in which one or more of the stops 118, 128 are integrally formed with the shaft 104, the wafers 102-i can be slidably coupled to the shaft 104 by pressing at least one of the stops 118, 128 through the wafer's opening, which can at least partially elastically deform the opening to permit passage of at least one of the stops 118, 128 therethrough. Once the one or more of the stops 118, 128 have been pressed through the opening, the opening can at least partially elastically recover, thereby resisting passage of the one or more stops 118, 128 back through the opening. Various other arrangements can be employed for providing stops on the shaft and/or for controlling and/or limiting translation of the wafers along the shaft.

The anchor 106 can include a helical member 132 coupled to the shaft 104. As shown in FIG. 1, the helical member 132 can be loosely wound such that adjacent turns of the helical member 132 do not contact one another. For example, the helical member 132 can resemble a corkscrew-type configuration. The anchor portion 106 can be engaged with tissue by rotating the anchor 106 about the axis of the helical member, thereby advancing the anchor 106 into tissue. Consistent with such an embodiment, the anchor 106 can resist pulling out from the tissue. The anchor 106 can be provided as an extension of the shaft 104 in the form of a helical configuration. Alternatively and/or additionally, the anchor 106 can be formed as a separate feature and can be coupled to the shaft 104, e.g., using mechanical fasteners, welding, adhesive, etc.

According to various alternative embodiments, the anchor portion 106 can include various configurations capable of being coupled to and/or otherwise attached to native coronary tissue. For example, the anchor portion 106 can include one or more prongs adapted to pierce coronary tissue and to, alone or in conjunction with other features, resist removal of the anchor portion from tissue. For example, the anchor portion 106 can include a plurality of prongs which can engage native coronary tissue.

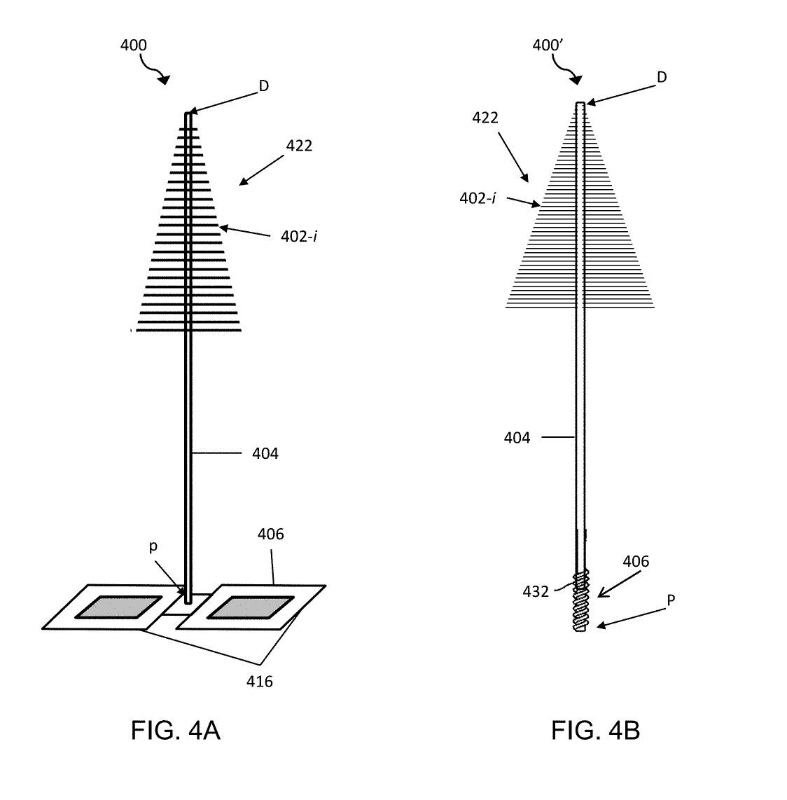

Further, the anchor portion can include features, without limitation, which facilitate attachment to the tissue by suturing. Examples of the features that can be used for facilitating suturing can include rings or openings, suture penetrable tabs, etc. (e.g., shown later in FIG. 4A). Various other anchor portions that can allow attachment or coupling to native coronary tissue can also suitably be employed in connection with a heart implant according to the present teachings.

Turning to FIG. 2, the mitral valve implant 100 is shown implanted within a heart 202. The mitral valve implant 100 can be disposed at least partially within the left ventricle 104 of the heart 102. As shown, the anchor portion 106 can be engaged with native coronary tissue within and/or adjacent to the left ventricle 204. The shaft 104, coupled to the anchor portion 106, can extend into the left ventricle 204. The shaft 104 can further extend at least partially within the mitral valve 208. For example, the shaft 104 can extend at least partially between the cusps of the mitral valve 208, and can also extend at least partially into the left atrium 206. The plurality of wafers 102-i forming a stacked array 122 of the mitral valve implant 100 can be positioned at least partially within the left ventricle 204 with the proximal portion P of the implant 100 disposed within the left ventricle 204 and the distal portion D positioned at least partially within and/or pointed towards the left atrium 206.

As the left ventricle 204 contracts, the pressure of blood in the left ventricle 204 can increase such that the blood pressure in the left ventricle 204 is greater than the blood pressure in the left atrium 206. Additionally, as the pressure of the blood in the left ventricle 204 initially increases above the pressure of the blood in the left atrium 206, blood can begin to flow towards and/or back into the left atrium 206. The pressure differential and/or initial flow of blood from the left ventricle 204 into the left atrium 206 can act against the stacked array of wafers 122 and can translate the wafers 102-i toward the left atrium 204. For example, pressurized blood within the left ventricle 204 can act against the bottom (proximal end Pₛ) of the stacked array of wafers 122 inducing sliding translation of the stacked array of wafers 122 along the shaft 104 toward the left atrium 206.

Illustrated in FIG. 2, the mitral valve implant 100 is shown in a closed position. In the closed position, the stacked array of wafers 122 can be translated toward and/or at least partially into the left atrium 206. At least a portion of the stacked array 122 can interact with, engage, and/or be positioned adjacent to at least a portion of the mitral valve 208. For example, at least a portion of at least one cusp of the mitral valve 208 can contact at least a portion of a sidewall of one or more wafers of the stacked array 122. Engagement between the stacked array 122 and the mitral valve 208 can restrict and/or prevent the flow of blood from the left ventricle 204 back into the left atrium 206.

In addition to the translation of the stacked array of wafers 122, the mitral valve 208 can also at least partially close around the stacked array of wafers 122, thereby also restricting and/or preventing the flow of blood from the left ventricle 204 to the left atrium 206. For example, as mentioned above, at least a portion of one or both of the cusps of the mitral valve 208 can contact at least a portion of the wafers 102-i. As the pressure of the blood in the left ventricle 204 increases, the pressure against the proximal end Pₛ of the stacked array of wafers 122 can increase. This increase in pressure against the bottom Pₛ of the stacked array can, in turn, increase the engagement between the stacked array 122 and the mitral valve 208.

Sliding translation of the stacked array 122 toward the left atrium 206 can at least partially be controlled and/or limited by the stop 118 coupled to the shaft 104. Additionally, translation of the stacked array of wafers 122 toward the left atrium 206 can be at least partially limited and/or controlled by engagement between the stacked array and the mitral valve 208. One or both of these restrictions on the translation of the stacked array can, in some embodiments, prevent the stacked array from passing fully into the left atrium 206. Furthermore, the diameter of the proximal portion Pₛ of the stacked array 122 can limit and/or restrict the movement of the stacked array 122 into the left atrium 206. Once the stacked array of wafers 122 has been positioned, the position of the stacked array of wafers 122 on the shaft 104 can be fixed, e.g. by frictional engagement between the stacked array of wafers 122 and the shaft 104 or using other coupling mechanisms.

Accordingly, the mitral valve implant 100 can be slidably translatable relative to the mitral valve 208 to reduce and/or eliminate regurgitation. Further embodiments of a mitral valve implant having axially translating wafers can be provided including various alternative wafers configurations. For example, in one embodiment a stacked array can be provided generally configured as a plurality of disc-shaped wafers. In the same manner, as illustrated embodiments of FIGS. 4-7 show, the disc-shaped wafers can translate along a shaft between an open position spaced from the mitral valve of the heart and closed position at least partially engaging the mitral valve and/or at least partially obstructing a flow of blood from the left ventricle to the left atrium. Implants employing wafers having various other geometries, such as spherical, oblong, etc., can also be employed. Furthermore, in addition to the slidably translatable stacked array depicted in FIG. 2, embodiments can be provided in which the stacked array is rotatably and/or pivotally translatable to engage and/or interact with at least a portion of the mitral valve. Further, in some embodiments, one or more of the wafers can be tilted relative to the shaft.

In the example shown in FIG. 2, the illustrated mitral valve implant 100 includes a single anchor portion 106 coupled to a proximal end (P) of the shaft 104. However, a mitral valve implant according to the present disclosure can include more than one anchor portion for securing the mitral valve implant to native coronary tissue. Additional anchor portions can be employed to provide more secure coupling of the valve implant to coronary tissue. Furthermore, more than one anchor portion can be employed to achieve more precise positioning of the valve implant and/or the wafers portion of the valve implant within the heart. For example, a replacement valve can include an anchor portion coupled to the proximal end of the shaft and another anchor portion coupled to the distal end of the shaft. Further, each end of the shaft can be coupled to native coronary tissue. The orientation of the shaft, and thereby the path of translation of the wafers, can be controlled by coupling each end of the shaft to native coronary tissue. Furthermore, the valve implant can include an anchor portion coupled to one end of the shaft and another anchor portion coupled to the shaft between the ends thereof.

The mitral valve implants described herein can be produced from a variety of suitable materials. Generally, such materials are biocompatible. Suitable materials can include, without limitation, biocompatible polymers, such as silicone, polyurethane, etc. Various metals can additionally be used in connection with a valve implant, such as titanium, stainless steel, etc. Additionally, biological materials and/or materials which can promote cellular ingrowth can also be used in connection with a valve implant described herein. Furthermore, various combinations of materials can be used herein, e.g., providing composite features and/or portions made from different materials. For example, the shaft can be formed from a biocompatible polymer or metal and the wafers can be formed from a polymeric material. Various additional and/or alternative combinations can also be employed herein.

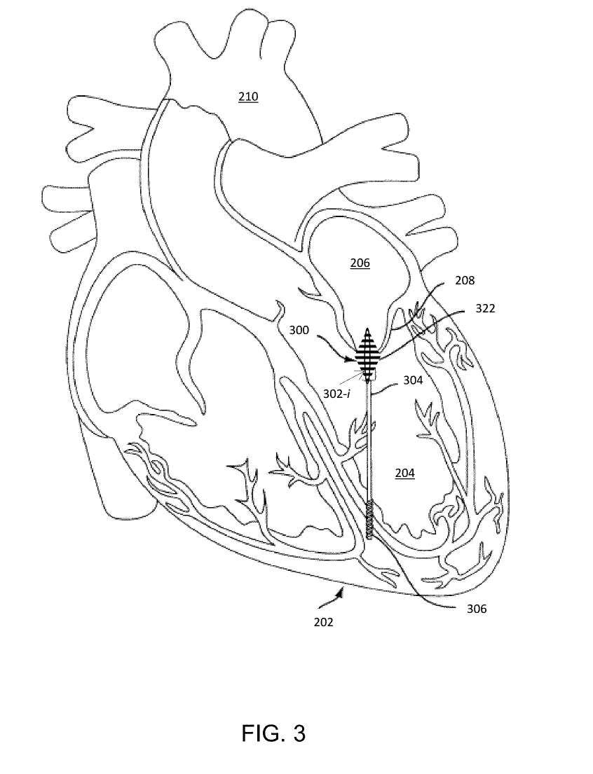

FIG. 3 illustrates a mitral valve implant 300 according to the embodiments described herein. The mitral valve implant 300 is shown after it has been implanted within the heart 202. As shown in FIG. 3, the mitral valve implant 300 can be positioned such that it extends at least partially into and/or through the mitral valve 208 between the left ventricle 204 and the left atrium 206. When the pressure of blood in the left atrium 206 is higher than the pressure of blood in the left ventricle 204, for example during contraction of the left atrium 206, the mitral valve 208 can be in an open condition/configuration. In the open configuration, blood can flow from the left atrium 206 through the mitral valve 208 and around the stacked array of wafers 322 and into the left atrium 204.

The anchor 306 can be engaged in native coronary tissue surrounding and/or defining at least a portion of the left ventricle 204. The stacked array of wafers 322 can be positioned extending at least partially between the mitral valve 208 by the shaft 304 extending between the anchor 306 and the stacked array of wafers 322. In related embodiments, the anchor 306 can be engaged in tissue surrounding and/or defining at least a portion of the left atrium. Further, the stacked array of wafers 322 can be positioned extending at least partially between the mitral valve 208.

FIG. 3 depicts the mitral valve implant 300 implanted in a heart 102 with the mitral valve 208 in a closed condition. The closed condition of the mitral valve 208 can occur when the pressure of blood in the left ventricle 204 is higher than the pressure of blood in the left atrium 206. As shown, when the mitral valve 208 is in a closed condition at least a portion of the mitral valve 208 can interact with, engage, and/or seal against the stacked array of wafers 322 of the mitral valve implant 300. The presence of the mitral valve implant 300 can enhance the closure of the mitral valve 208 so as to provide an adequate seal, to permit ejection of blood from the ventricle 204 through the aorta 210 and prevent and/or reduce mitral regurgitation.

The stacked array of wafers 322 can be shaped to facilitate the flow of blood from the left atrium 206 to the left ventricle 204 when the mitral valve 208 is open. The stacked array of wafers 322 can have a generally streamlined shape, allowing for the flow of blood around the stacked array of wafers 322. For example, the stacked array of wafers 322 can have a generally diamond shape. In other embodiments, the stacked array 322 can have a generally cylindrical, prismatic, etc. shape, without limitation.

The performance of the mitral valve implant 300 for reducing and/or eliminating mitral valve regurgitation can be, at least in part, related to the positioning of stacked array of wafers 322 relative to the mitral valve 208. In an embodiment consistent with this aspect, the wafers 302-i can be fixed (e.g., non-slidable) on the shaft 304. The mitral implant can be positioned such that the stacked array of wafers 322 extends at least partially within the mitral valve 208. The size of the stacked array of wafers 322 allows for variations in size of a patient's heart, such that the position of the stacked array of wafers 322 can accommodate any differences once the anchor 306 is in the heart 202.

The illustrated and described embodiments of the mitral valve implant include a stacked array of wafers coupled to a shaft 304. The shaft 304, as used herein, can be a rigid or semi-rigid. Alternatively or additionally, the shaft 304 can be a flexible member. The shaft 304 can be formed of a flexible material, such as a polymer, and can be in the form of a wire or filament. In some embodiments, such a flexible shaft 304 can be coupled to at least two anchor portions (see, e.g., FIG. 4A). For example, the flexible shaft 304 can extend through the openings in the wafers. An anchor can be coupled to the flexible shaft 304 on one or each side of the wafers 302-i. For example, the flexible shaft 304 can be used to position the wafers 302-i relative to the mitral valve 208 and can be anchored or otherwise coupled to the left ventricle 204 and/or to the left atrium 206, on one or both sides of the wafers 302-i.

A mitral valve implant including a flexible shaft can be employed in implementations including those in which the stacked array of wafers 322 can slidably translate along the flexible material of the shaft 304. In a related embodiment, the wafers 302-i can be non-slidably coupled to the flexible shaft 304. The flexible shaft 304 can have a length which permits the wafers to move toward and away from the mitral valve.

A mitral valve implant including a flexible shaft can also suitably be employed with implementations having stationary (non-translatable) wafers. The wafers 302-i can be generally non-slidably coupled to the flexible shaft 304. The flexible shaft 304 can be coupled to an anchor that engages native coronary tissue, e.g., via the anchor portions (not shown in FIG. 3, see for example, FIG. 4A), etc., on one or both sides of the wafers. Coupling the flexible material on either side of the wafers can generally maintain the wafers in a position within and/or relative to the mitral valve 208.

The heart valve implants according to the present disclosure can be implanted using a variety of surgical and/or non-surgical procedures and/or minimally invasive surgical procedures. A surgical implantation procedure can include, for example, an open heart procedure in which the implant can be directly placed into the heart and manually positioned relative to the heart valve.

A heart valve implant consistent with the present disclosure can also advantageously be implanted using less invasive procedures. For example, the heart valve implant can be implanted using a percutaneous procedure. A suitable percutaneous implantation procedure can include a catheterization procedure. For example, if used as a mitral valve implant in a percutaneous catheterization procedure, the mitral valve implant can be delivered to the heart using a catheter inserted into a vein or artery or directly into the heart itself (via the apex), depending upon the desired delivery site, and into the left atrium or the left ventricle. For example, the mitral valve implant can be delivered via a transceptal approach, in which the catheter is inserted, e.g., via a vein, into the right atrium. The catheter can then pass through a puncture between the right atrium to the left atrium and further through the mitral valve to the left ventricle, if desired. Generally, according to a catheterization procedure, the vein or artery can be accessed through a percutaneous incision or puncture. A catheter carrying the mitral valve implant can be introduced into the vein or artery through the incision or puncture. The catheter and mitral valve implant can be passed through the vein or artery into the heart. Once in the heart, the mitral valve implant can be deployed from the catheter and positioned within and/or between the left ventricle and the left atrium.

At least a portion of the heart valve implants described herein can be collapsible and/or reducible in volume to facilitate percutaneous and/or transluminal delivery. In such a manner, the wafers of the mitral valve implant can be collapsible, which can be reduced in volume and/or reduced in maximum diameter during delivery to the heart and/or during placement and/or attachment of the anchor to native coronary tissue. After delivery to the heart, the wafers can be expanded, inflated, and/or otherwise increased in volume or size. Accordingly, the mitral valve implant can be delivered to an implantation site via a smaller diameter catheter, and/or via smaller vessels, than would otherwise be required.

The wafers can be formed from a resiliently deformable material, such as an elastomer, which can be elastically deformed under stress. The wafers can elastically recover when the stress is removed. In such an embodiment, the wafers can, for example, be deformed from an expanded configuration to a collapsed condition and loaded into a catheter delivery system. After delivery to an implant site, the wafers can be deployed from the catheter delivery system, thereby removing the deforming stress from the wafers. Once the deforming stress is removed, the wafers can resiliently recover back to the expanded configuration.

FIGS. 4A-4B are examples of mitral valve implants 400, 400′, according to the embodiments disclosed herein. The example mitral valve implants 400, 400′ include a shaft 404 having a proximal end (P) and a distal end (D). A plurality of wafers 402-i are disposed or coupled to the shaft 404 at or near the distal end (D) of the shaft 404. The plurality of wafers 402-i form a stacked array of wafers 422.

In FIG. 4A, the wafers 402-i are stacked substantially parallel to each other and have substantially the same spacing between the wafers 402-i. Wafers 402-i are also coupled to shaft 404 such that they are substantially perpendicular to the shaft. In FIG. 4B, the wafers 402-i are also stacked substantially parallel to each other and have substantially the same spacing between the wafers 402-i. However, the wafers 402-i are spaced closer together than the wafers 402-i illustrated in FIG. 4A.

In the embodiments illustrated in FIGS. 4A-4B, the cross-sectional diameter of each of the wafers 402-i in the stacked array 422 is variable, and the size (e.g., diameter) of each wafer 402-i is different. The stacked array of wafers 422 of FIGS. 4A and 4B have a generally triangular shape, with the diameters of the wafers progressively decreasing from a maximum value to a minimum value close to the distal end of the shaft (D). Once implanted in the heart (not shown, see, e.g., FIG. 3), a portion of the tapered stacked array of wafers 422 can be disposed above the mitral valve in the left atrium. The proximal portion can be disposed below the mitral valve in the left ventricle. The size and shape of each of the wafers 402, which determines the size and shape of the stacked array 422, can be configured for placement within the heart valve (e.g., a mitral valve), so as to at least partially restrict blood flow through the heart valve during systole. Various sizes and shapes of wafers 402-i (and, thereby, various sizes and shapes of stacked array of wafers 422) can be utilized. The size and shape of the wafers 402-i on the heart valve in which the implant is implanted.

As shown in FIG. 4B, an anchor 406 can be coupled to the shaft 404 at or near the first end. The anchor 406 can be used to secure the mitral valve implant 400′ to a patient's heart. As noted, the mitral valve implant 400, 400′ can generally include a plurality of disc-shaped wafers 402-i coupled to a shaft 404. The wafers 402-i can be coupled to the shaft 404 in a stationary fashion, e.g., the wafers can be coupled to the shaft in a non-slidable manner. Generally, the wafers 402-i can be maintained at a generally fixed position on the shaft 404. The mitral valve implant 400, 400′ can be implanted in the heart such that the anchor 406 and the shaft 404 can maintain the wafers 402-i in a desired position relative to various elements/features of the coronary anatomy. According to one aspect, the anchor 406 and the shaft 404 can maintain the wafers 402-i positioned extending at least partially within the mitral valve.

The wafers 402-i can be maintained in a stationary position on the shaft 404 in various ways. For example, wafers 402-i can be integrally formed on the shaft 404. Additionally and/or alternatively, the wafers 402-i can be adhesively bonded, welded, staked, and/or mechanically fastened to the shaft 404. As noted with reference to FIG. 1, the shaft 404 can include one or more stops or features (not shown in FIG. 4) which can prevent and/or limit translation of the wafers along the shaft. For example, the shaft 404 can include a stop (not shown in FIG. 4, see, e.g., FIG. 1) closely positioned on either end of the stacked array of wafers 422, thereby restricting movement of the wafers. The stops can be fixed and/or can be adjustable along the shaft 404. Various other configurations and/or arrangements can be employed for coupling the wafers 402 in a stationary manner with respect to the shaft 404. For example, as noted on FIG. 1, one or more coupling members 142 can be used to couple each wafer 102-i to the shaft 142.

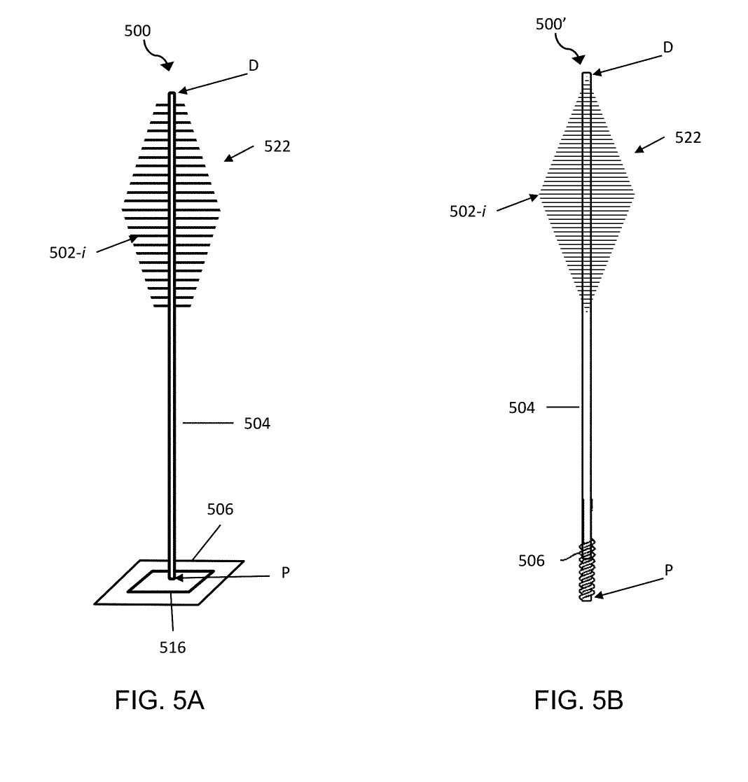

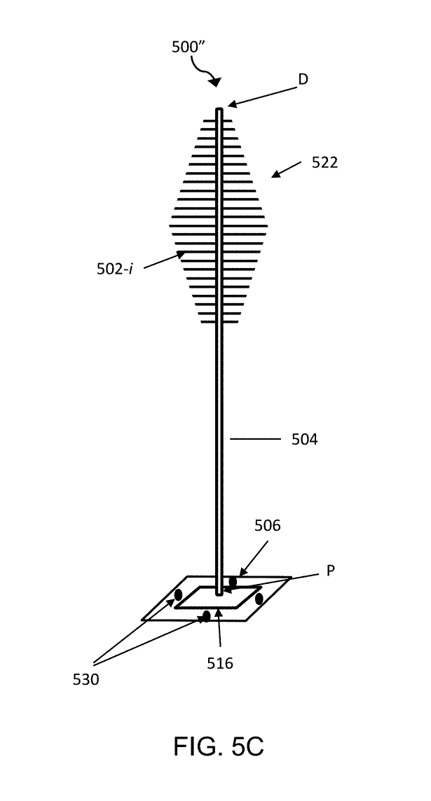

Any number of anchors known in the art can be used to secure the mitral valve implant 400, 400′ to the heart, such as barbs, helical members, anchor pads, etc. For example, anchor 406 of FIG. 4A is coupled to a first end of shaft 404. The anchor 406 can have one or more anchor pads 416 that can be used to secure the implant 400, 400′ to a wall of the heart. The anchor pads 416 can allow for tissue ingrowth and/or one or more sutures to secure the anchor to heart tissue. Consistent with related embodiments, the anchor 406 can be formed as a separate feature and can be coupled to the shaft 404, e.g., using mechanical fasteners, compression fittings, welding, adhesive, etc. The anchor pads 416 can be placed on an inside surface of the heart, an outside surface of the heart, or both. In instances where anchor pads 416 are secured to both an inside and outside surface of the heart, or to an outside surface only, the shaft 404 can extend through a heart wall (e.g., the left ventricle). The anchor pads 416 (e.g., tabs or rings) can provide suturing features (not shown, see, e.g., FIG. 5C) through which sutures can pass to secure the anchor to coronary tissue. For example, as shown in FIG. 5C, the anchor pad 416, 516 can include a plurality of holes 530 that allow suturing the anchor pad 416, 516 to the heart tissue.

In FIG. 4B, anchor 406 includes a helical member 432 having a corkscrew shape coupled to the shaft 404. The helical member 432 can be loosely wound such that adjacent turns of the helical member 432 do not contact one another, for example resembling a corkscrew-type configuration. The helical member 432 of the anchor 406 can be engaged with coronary tissue (not shown, see, e.g., FIG. 2) by rotating the anchor 406 about the axis of the helix 432, thereby driving the anchor 406 into native coronary tissue. Once the anchor 406 has been engaged with native coronary tissue, the anchor 406 can resist axial pull-out from the tissue. The anchor 406 can be provided as an extension of the shaft 404 wound in a helical configuration. Consistent with related embodiments, the anchor 406 can be formed as a separate feature and can be coupled to the shaft 404, e.g., using mechanical fasteners, welding, adhesive, etc. The anchor 406 can additionally and/or alternatively be provided having various features and/or configurations.

FIGS. 5A-5C illustrate example embodiments of the mitral valve implant 500, 500′, 500″. The mitral valve implant 500, 500′, 500″ can have a shaft 504 with a proximal end (P) and a distal end (D). An anchor 506 is coupled to the shaft 504 at or near the proximal end (P) thereof and arranged to secure the mitral valve implant 500, 500′, 500″ to a patient's heart (not shown, see, e.g., FIG. 2). A plurality of wafers 502-i are disposed or coupled to the shaft 504 at or near the distal end (D) of the shaft 504. The plurality of wafers 502-i form a stacked array of wafers 522.

In FIG. 5A, the wafers 502-i are stacked substantially parallel to one another and arranged to have substantially the same spacing between the wafers 502-i.

In FIG. 5B, the wafers 502-i are also stacked substantially parallel to each other and have substantially the same spacing between the wafers 502-i, but the wafers are spaced closer together than the wafers illustrated in FIG. 5A.

In the embodiments illustrated in FIGS. 5A-5C, the cross-sectional diameter of the stacked array of wafers is variable, i.e., the width (diameter) of each wafer varies. The stacked array of wafers 522 of FIGS. 5A-5C have a generally symmetrical shape, forming a diamond-like shape. The size and shape of each of the wafers 502-i, determines the size and shape of the stacked array 522. This shape (of the stacked array 522) is configured for placement within a heart valve (e.g., a mitral valve, not shown, see, e.g., FIG. 2), so as to restrict blood flow through the heart valve during systole.

As noted previously, the cross-sectional shape of the stacked array of wafers can be sized for placement within a heart valve, such as a mitral valve or any other heart valve in which the implant may be implanted. The shapes illustrated in FIGS. 5A-5C are provided as examples, and other shapes, for example, round, oval, pear-shaped, elliptical, hour-glass shaped, and heart shaped, can be used in the heart valve implants described herein. These shapes can have a variable cross-sectional diameter, in which the wafers have varying sizes. These shapes can also have a substantially equal cross-sectional diameter, in which the wafers have about equal widths, for example, in rectangular and square shaped arrays.

The wafers can form a compliant (e.g., a smooth) surface with which heart valve leaflets can engage. For example, the wafers can comprise a biocompatible polymer, a shape memory material, or a combination thereof. Shape memory materials are known in the art and can comprise, for example, an alloy, a polymer, or a combination thereof. Nitinol is an example of a shape memory alloy. Examples of shape memory polymers are polyurethanes, polyurethanes with ionic or mesogenic components made by prepolymer method, block copolymers of polyethylene terephthalate (PET) and polyethyleneoxide (PEO), block copolymers containing polystyrene and poly(1,4-butadiene), and an ABA triblock copolymer made from poly(2-methyl-2-oxazoline) and polytetrahydrofuran. Other polymers, for example, can include polysiloxanes (silicones) and polyethers.

The wafers can be collapsed and/or otherwise deformed from an expanded configuration. The collapsed and/or deformed wafers can maintain the collapsed and/or deformed configuration after the initial deforming stress is released. The wafers can subsequently be returned to the expanded and/or operable configuration, for example, by heating the wafers above an activation temperature of the shape memory material, which can induce recovery of the shape memory material to a pre-deformed shape. The activation temperature inducing recovery of the deformed wafers can be provided by the body temperature of the patient receiving the mitral valve implant. Alternatively or additionally, heat for activating recovery of the shape memory material can be provided by a heating element coupled to the wafers and/or a heating element delivered through a catheter. Heat activation can also be provided by irradiating the shape memory material using suitable radiation techniques, such as, with microwaves, IR light, etc.

The wafers can also be collapsed and/or deformed to facilitate delivery of the implant to the desired site, e.g., via a transluminal and/or a surgical procedure. The wafers can subsequently be recovered to an expanded configuration. For example, when using a thermally activated shape memory material, recovery of the shape memory wafers can be accomplished by heating the wafers to, or above, an activation temperature. Heat for activating the shape memory material can be provided by the body temperature of the subject receiving the mitral valve implant, and/or from an external source, e.g., via the catheter, etc.

Referring back to FIGS. 5A-5C, the wafers can be slidably coupled to the shaft. For example, each wafer can have a central channel (e.g., opening 112, shown in FIG. 1) to receive the shaft 504 and advance over the shaft 504. One or more coupling members (e.g., coupling member 142, shown in FIG. 1) can couple each wafer to the shaft 504. The wafers can coupled directly with the shaft 504. In some embodiments, the wafers can be attached to the shaft 504 such that they are not slidably attached. For example, as noted above, the wafers can be fixedly attached to the shaft.

As shown in FIGS. 5B-5C, the anchor 506 of mitral valve implant 502, 503 can have an anchor pad 516 that can be used to secure the implant 500 to a wall of the heart (not shown, see, e.g., FIG. 2). The anchor pad 516 can allow for tissue ingrowth and/or one or more sutures 530 to secure the anchor to heart tissue. The anchor pad 516 can be placed on an inside surface of the heart or an outside surface of the heart. In instances where anchor pad 516 is secured to an outside surface, the shaft 504 extends through a heart wall (e.g., the left ventricle). As shown in FIG. 5C, the anchor pad 516 (e.g., a tab or ring) can provide suturing features 530 through which one or more sutures can pass to secure the anchor 504 to coronary tissue.

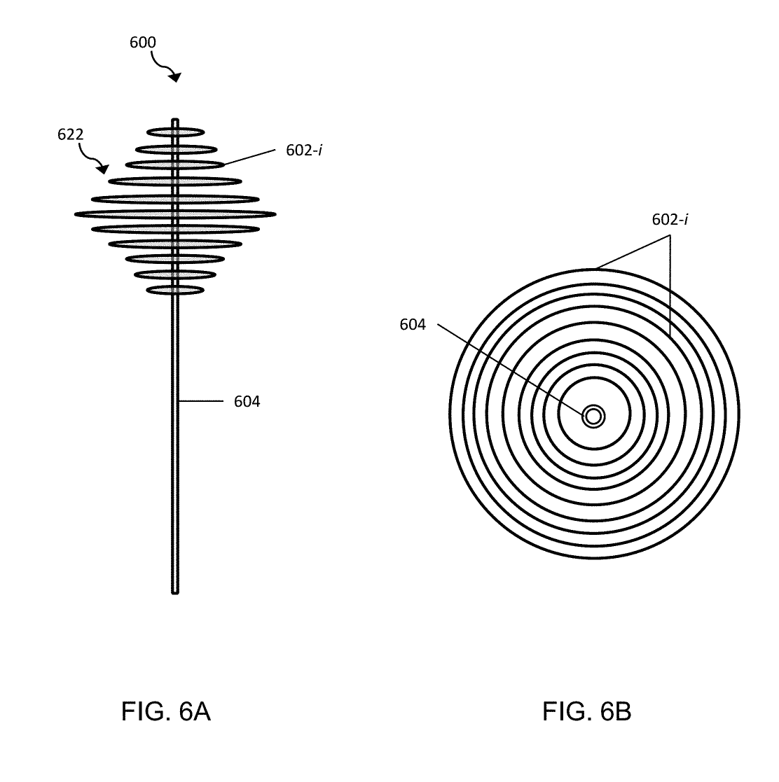

FIG. 6A is a perspective view of an example heart valve implant 600 according to embodiments disclosed herein. In this example, wafers 602-i have a substantially circular or disc-like shape and are each coupled to shaft 604. The plurality of wafers 602-i form a stacked array of wafers 622 having two wafers at each end with a minimum diameter and a wafer at the middle with a maximum diameter. The spacing between wafers 602 can be substantially equal, as illustrated in FIG. 6A, or can vary.

FIG. 6B is a top view of the heart valve implant 600 shown in FIG. 6A. The shaft 604 is disposed generally along a central axis. Wafers 602-i are generally circular in shape and are configured to be sized for at least partial placement in a heart valve.

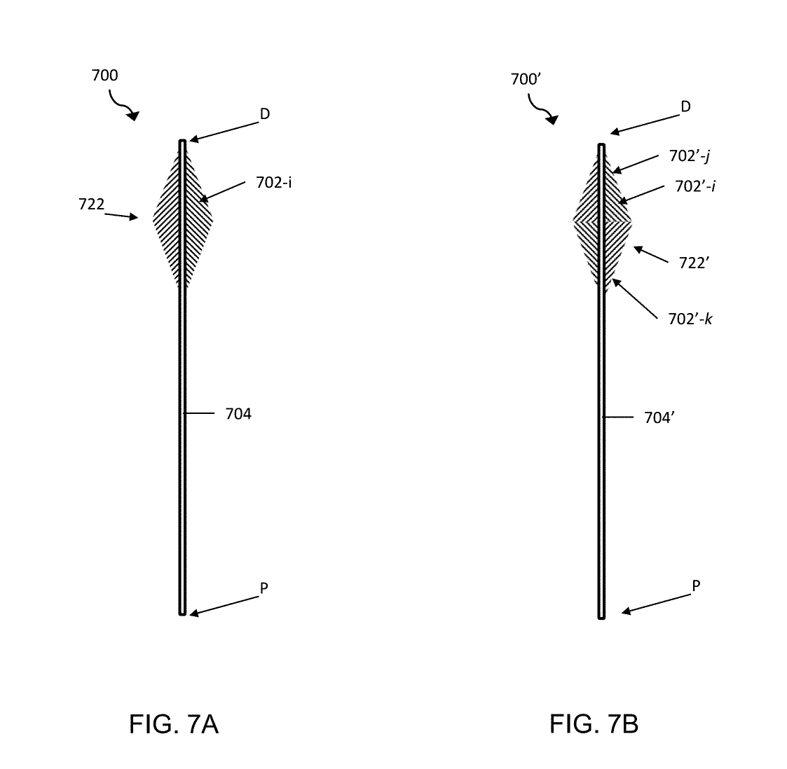

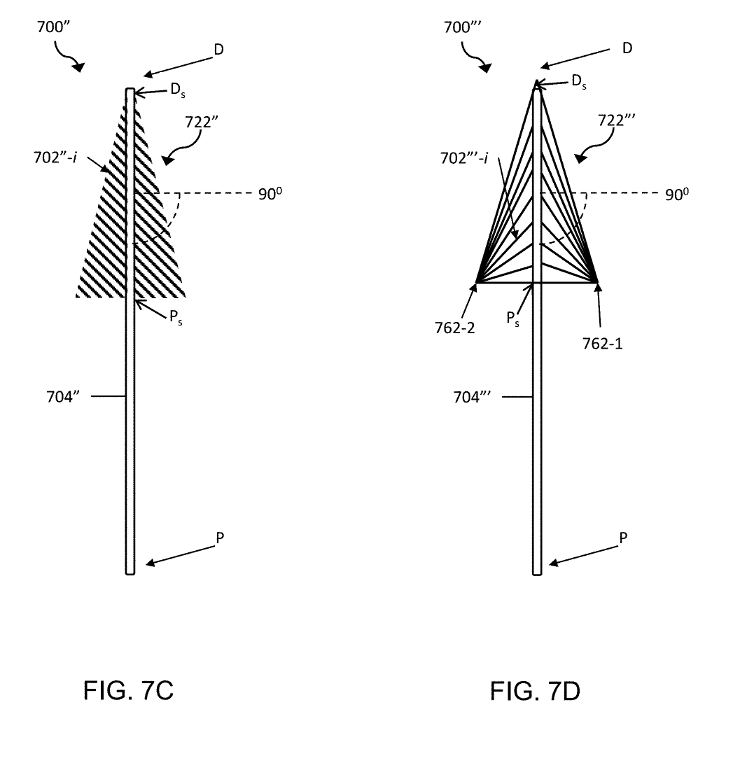

FIG. 7A illustrates a heart valve implant 700 having a plurality of wafers 702-i forming a stacked array 722. Similar to the stacked array 522 illustrated in FIG. 5A-5C, the cross-sectional shape of the stacked array of wafers 722, shown in FIG. 7A, can be diamond-shaped, having two wafers at both ends with a minimum diameter and a wafer in the middle with a maximum diameter. Further, each of the plurality of wafer 702-i can be coupled to a shaft 704 at a non-perpendicular angle (i.e., less than 90°) relative to the shaft 704. The shaft 704 can have a proximal end (P) and a distal end (D). The wafers 702-i can be configured such that, once implanted, they are directed to or angled towards the proximal end (P) of shaft 704 (e.g., if used as a mitral valve implant, they are angled downwards towards the left ventricle in a “V” shape formation). For example, each one of the wafers 702-i can be angled at any of less than 90°, less than 80°, less than 70°, less than 60°, less than 50°, less than 40°, less than 30°, less than 20°, or less than 10° relative to shaft 704. When used as a mitral valve implant, in such a configuration, the implant 700 can allow blood to more easily flow from an atrium to a ventricle during diastole. During systole, blood flow is at least partially restricted back through the heart valve because of contact between a portion of a valve leaflet with the stacked array. Also, the angle of and space between each of the wafers 702-i forming the stacked array 722 further prevents valve regurgitation even if valve leaflets do not completely abut against the stacked array 722.

In the example shown in FIG. 7B, the heart valve implant 700′ has a plurality of wafers 702′-i forming a stacked array 722′. Similar to the stacked array 722 illustrated in FIG. 7A, the cross-sectional shape of the stacked array of wafers 722′ can be diamond-shaped, having two wafers at both ends with a minimum diameter and a wafer in the middle with a maximum diameter. Each of the wafers 702′-i can be coupled to the shaft 704′ at a non-perpendicular angle relative to shaft 704′. Wafers 702′-i can be “V” shaped and oriented in opposite directions. Specifically, some of the wafers (e.g., wafer 702′-j) can be configured such that they are directed to or angled towards the proximal end (P) of the shaft 704′(e.g., angled towards the left ventricle when used as a mitral valve implant). Some of the other wafers (e.g., for example wafer 702′-k) can be configured such that they are directed to or angled towards the distal end (D) of shaft 704′ (i.e., angled towards the left atrium). For example, each one of the wafers 702′-i can be oriented or coupled with shaft 704′ at a non-90 degree angle. Specifically, wafers 702′-i can be angled at more or less than 90°. In some embodiments, the wafers 702′-i are angled at less than 80°, less than 70°, less than 60°, less than 50°, less than 40°, less than 30°, less than 20°, or less than 10° relative to shaft 704′. In such a configuration, the heart valve implant 700′ (e.g., when used as a mitral valve implant) can allow blood to more easily flow from an atrium to a ventricle during diastole. During systole, blood flow is at least partially restricted back through the heart valve because of contact between a portion of a valve leaflet with the stacked array. Also, the angle of and space between each of the wafers forming the stacked array further prevents valve regurgitation even if valve leaflets do not completely abut against the stacked array.

Referring to FIG. 7C, the heart valve implant 700″ is yet another embodiment of the heart valve implant disclosed herein. The mitral valve implant 700″ can have a plurality of wafers 702″-i at a non-perpendicular angle relative to the shaft 704″ forming a stacked array of wafers 722″. The cross-sectional shape of stacked array can be triangular or tree-shaped. Similar to FIG. 7A, wafers 702″-i are coupled to the shaft 704″ such that they are directed to or angled towards the proximal end (P) of the shaft 704″, (e.g., angled downwards towards the proximal end (P)). For reference, a right angle is illustrated with dotted lines in FIG. 7C. The stacked array of wafers 722″ has a distal portion (Dₛ) that is configured to at least partially sit above a heart valve (e.g., in a left atrium) and an proximal portion (Pₛ) that is configured to at least partially sit below a heart valve (e.g., in a left ventricle).

The wafers 702-i and wafers 702″-i shown in FIG. 7A and FIG. 7C are arranged such that each wafer 702-i, 702″-i is coupled to the shaft 704, 704″ at approximately the same angle (e.g., an angle less than 90°). However, in some embodiments, a heart valve implant can have a plurality of wafers that are each coupled to a shaft at a range of angles, for example, between 0 and 180° (e.g., FIG. 7B). For example, the plurality of wafers forming a stacked array can be coupled to the shaft from about 10° to about 90°, from about 20° to about 90°, from about 30° to about 90°, from about 40° to about 90°, from about 20° to about 80°, from about 30° to about 70°, or from about 40° to about 60° relative to the shaft.

FIG. 7D illustrates another embodiment of the heart valve implant 700′″. The implant 700′″ includes a plurality of wafers, each of which 702′″-i varies in size and angle at which the wafer 702′″-i is coupled to the shaft 704′″. The plurality of wafers 702′″-i form a stacked array 722″. The cross-sectional shape of stacked array 722′″ can be triangular or tree-shaped. The stacked array of wafers 722′″ has a distal portion (Dₛ) that is configured to at least partially sit above a heart valve (e.g., in a left atrium) and a proximal portion (Pₛ) that is configured to at least partially sit below a heart valve (e.g., in a left ventricle). The angle at which each wafer 702′″-i is coupled to the shaft 704′″ gradually increases from the distal end to the proximal end. The wafers at the tapered/distal end (Dₛ) of the stacked array of wafers 722′″ have a non-zero angle of coupling, which increases to about 90 degrees at the enlarged/proximal end (Pₛ) of the stacked array of wafers 722″. Also, the size of each wafer 702′″-i gradually decreases from the distal end (Dₛ) of the stacked array of wafers 722′″ to the proximal end (Pₛ) of the stacked array of wafers 722″.

In the example shown in FIG. 7D, each wafer 702′″-i is generally directed towards the proximal end (Pₛ) of stacked array of wafers 722″, and ends at or near the enlarged ends/the proximal end (Pₛ) of the stacked array of wafers 722″. Each of the plurality of wafers 702′″-i can be coupled together on the proximal end (Pₛ) of the stacked array of wafers 722″. For example, as shown in FIG. 7D, the wafers 702′″-i are coupled together at two points 762-1, 762-2 on the proximal end (Pₛ) of the stacked array of wafers 722″.

FIG. 8 illustrates a mitral valve implant 800, having a shaft 804 with a proximal end (P) and a distal end (D). At the distal end (D) of the shaft 804 are a plurality of wafers 802-i forming a stacked array 822. Each of the plurality of wafers 802-i is coupled to the shaft 804 such that the wafer 802-i is substantially perpendicular to the shaft 804. An anchor 806 is coupled to a proximal end (P) of the shaft 806. Although a cork-screw type anchor 806 is illustrated in FIG. 8, any suitable anchor can be used with the shaft 804 to secure the mitral valve implant 800 to a patient's heart (not shown).

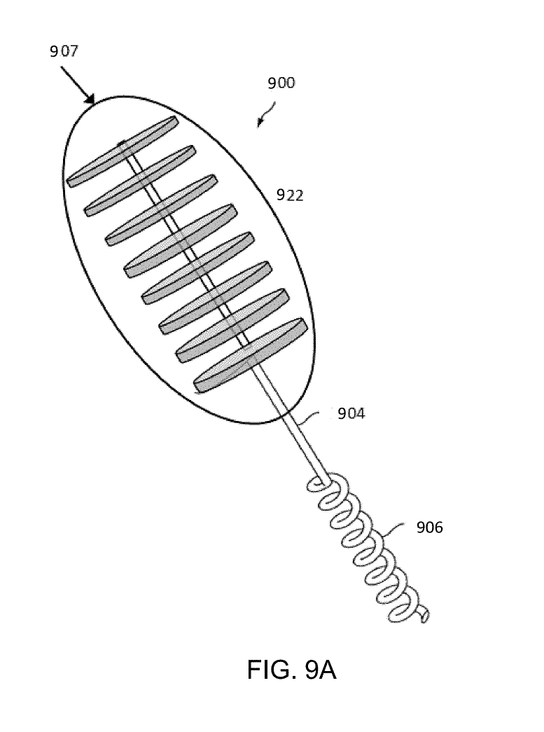

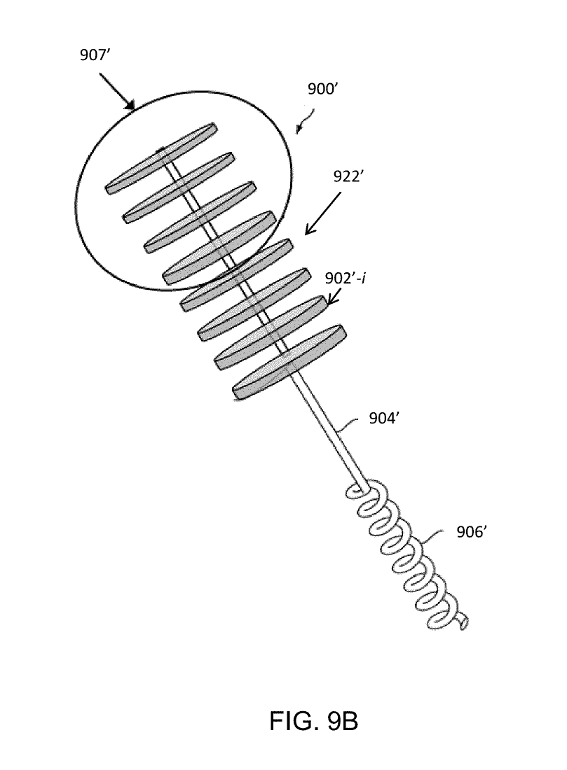

FIG. 9A illustrates heart valve implant system 900, in which an inflatable balloon 907 completely surrounds the stacked array of wafers 922. FIG. 9B schematically illustrates a heart valve implant 900′, in which an inflatable balloon 907′ partially surrounds a portion or portions of the stacked array of wafers 922′. The heart valve implant 900′ can have a plurality of wafers 902′-I coupled to a shaft 904 and further having a balloon 907′, which covers a subset of the wafers 902′-i. Similar to the previous embodiments, the implant system 900′, shown in FIG. 9B, includes an anchor 906 in the form of a cork-screw.

Alternatively or additionally, the balloon 907′ can surround a portion or portions of each one of the wafers 902′-i. The balloon 907′ can be configured to be inflated with a fluid, e.g., water or saline.

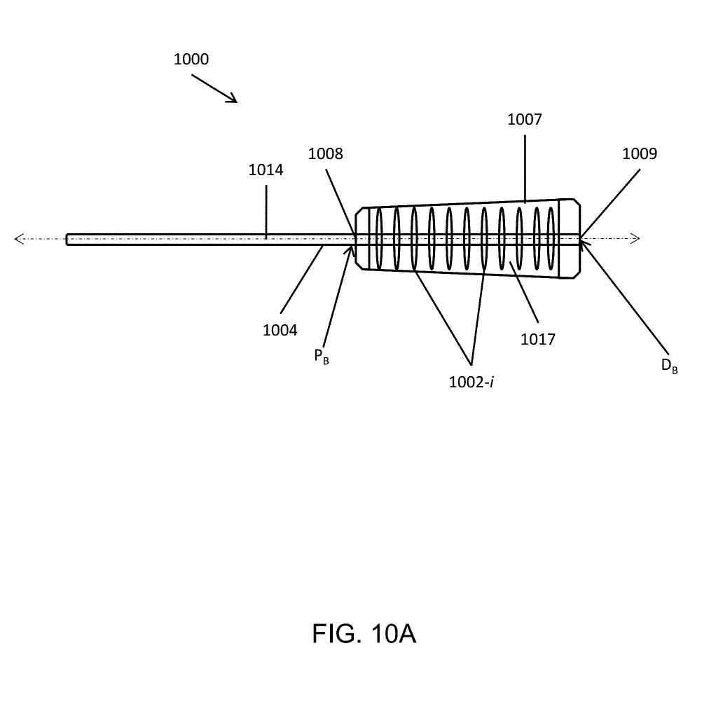

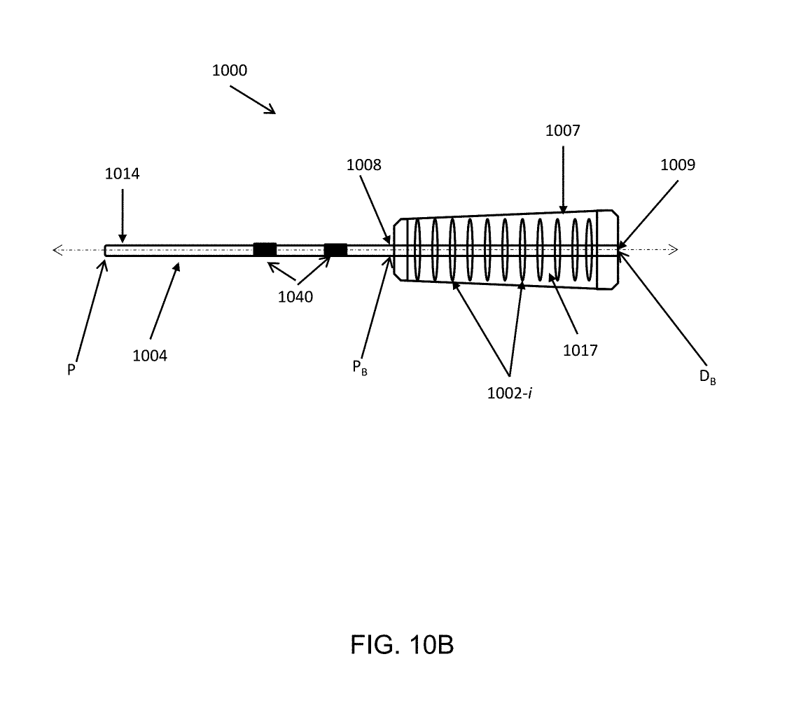

FIG. 10A illustrates an example of a mitral valve implant 1000. Implant 1000 includes a shaft 1004 and an inflatable balloon 1007. The balloon 1007 comprises a proximal end (P_{B}) and a distal end (D_{B}). The distal end of the balloon D_{B} is furthest from an opening 1008. The proximal end of balloon 1007 is at or near the opening 1008. In this example, the balloon 1007 is shown as completely surrounding wafers 1002-i, so that each wafer is within a spacer cavity 1017 defined by balloon 1007. However, in other embodiments, the balloon 1007 can surround one or some of the wafers 1002-i. the wafers 1002-i can provide internal structural support for the balloon 1007. The incorporation of a balloon surrounding some or all of the wafers can be beneficial for use in patients whose current medical treatment and condition, or blood flow around the spacer, may be insufficient to allow for non-turbulent flow around and between wafers. Non-turbulent flow may lead to the creation of blood clots. Surrounding the wafers with a balloon can aid in reducing the surface area of the wafer exposed directly to the blood and allow for smoother and non-turbulent blood flow.

FIG. 10B schematically illustrates a heart valve implant 1000 having one or more radiopaque markers 1040. The one or more radiopaque markers 1040 can be attached to the shaft 1004 and positioned at or near the proximal end P_{B} of the balloon 1007. Additionally or alternatively, the one or more radiopaque markers 1040 can be positioned at or near the distal end D_{B} of the balloon 1007. Further, the one or more radiopaque markers 1040 can be positioned at or near the proximal P_{B} and distal D_{B} ends of the balloon 1007.

Additionally and/or alternatively, the one or more radiopaque markers 1040 can be incorporated in the bodies of the wafers 1002-i to aid in the visualization of the movement of the implant 1000 during insertion and use. As will be appreciated by one of skill in the art, the one or more radiopaque markers 1040 can assist a physician in performing the methods described herein. For example, using known techniques (e.g., x-ray, fluoroscopy, etc.), a physician can use the radiopaque biomarkers 1040 to confirm correct placement and/or operation of the implant 1000.

The shaft 1004 can include a lumen 1014, which is in fluid communication with the spacer cavity 1017. For example, the shaft 1004 can extend to at least a proximal end (e.g., at or near opening 1008) of the balloon 1007. Alternatively and/or additionally, the shaft 1004 can extend through a proximal end P_{B} of the balloon 1007. Further, as illustrated in FIG. 10B, the shaft 1004 can be attached to the distal end D_{B} of balloon 1007 and extend through the proximal end P_{B} of the balloon 1007.

Any or all of the portions of the implants described herein can be formed from any biologically acceptable material. For example, materials such as Elast-Eon™ material can be used. At least the walls of balloon 1007 can be formed of a resiliently deformable biologically acceptable material.

The first (e.g., proximal P_{B}) end of the wall of balloon 1007 can be coupled, mounted, integrally formed with or otherwise secured to a portion of the shaft 1004. The implant 1000 can also include an opening 1008, proximate to the point of connection of the balloon with the shaft 1004. This opening 1008 can fluidly connect the lumen 1014 of the shaft 1004 with the cavity 1017 of balloon 1007. This connection can be used to direct a fluid (such as, but not limited to, saline or the like) through the lumen of the shaft into a the balloon cavity 1017 (e.g., from an inflation device (not shown)). Any suitable inflation device known in the art can be used. For example, the inflation device can be a syringe assembly. The opening 1008 can be a component (e.g., an integral part) of the balloon 1007 and/or can include an extension of the shaft 1004.

The cavity 1017 can be defined by the opening 1008 and the walls of the balloon 1007. The distal end D_{B} of the balloon 1007 can include an end plug 1009 that seals the distal end D_{B} of balloon 1007. Alternatively or additionally, the distal end D_{B} of balloon 1007 can be formed of a continuous piece of material such that the spacer cavity 1017 is naturally sealed at the distal end of balloon 1007.

A surgeon may selectively expand and/or retract the balloon 1007 and the spacer cavity 1017 by injecting and/or withdrawing an expansion or inflation medium into and from the spacer cavity 1017 (e.g., via lumen 1014). Once the spacer cavity 1017 is inflated to a desired degree, the degree of inflation can be maintained by an inflation device, which can be configured to limit or prevent the withdrawal of expansion or inflation medium from the spacer cavity 1017 by plugging or backstopping the lumen 1014 at a proximal end (P) of the shaft 1004.

Although, the implants described herein have been disclosed in the context of a mitral valve implant, an implant consistent with the present disclosure can also suitably be employed in other applications. For example, the implants described herein can be used with other valves of the heart, etc. For example, the size of the implant and/or the size of the wafers can be adjusted to configure the implant for use with a different heart valve (e.g., the aortic valve). The present disclosure should not, therefore, be construed as being limited to use for reducing and/or preventing regurgitation of the mitral valve.

Other features and aspects of the various embodiments can also suitably be combined and/or modified consistent with the present disclosure. The disclosure herein should not, therefore, be limited to any particular disclosed embodiment, and should be given full scope of the appended claims.

What is claimed:

1. A heart valve implant comprising:
a shaft having a first end and a second end;
an anchor coupled to the first end of the shaft, the anchor configured to secure the heart valve implant to a patient's heart; and
a plurality of wafers, wherein each of the plurality of wafers is coupled to the second end of the shaft to form a stacked array of wafers.

2. The heart valve implant of claim 1, wherein the stacked array of wafers is configured to at least partially restrict a flow of blood through the heart valve during systole upon contact with at least a portion of a valve leaflet of the heart valve.

3. The heart valve implant of claim 2, wherein the stacked array of wafers comprises a compliant surface.

4. The heart valve implant of claim 1, further comprising an inflatable balloon, wherein said balloon surrounds at least a portion of the plurality of wafers.

5. The heart valve implant of claim 4, wherein the balloon is at least partially inflated with a fluid.

6. The heart valve implant of claim 1, wherein one or more of the plurality of wafers comprises a biocompatible polymer.

7. The heart valve implant of claim 6, wherein the biocompatible polymer comprises a polyurethane, a polyethylene terephthalate (PET) and polyethyleneoxide (PEO) block copolymer, a polystyrene and poly(1,4-butadiene) block copolymer, a triblock copolymer made from poly(2-methyl-2-oxazoline) and polytetrahydrofuran, a polysiloxane, a polyether, or a combination thereof.

8. The heart valve implant of claim 1, wherein one or more of the plurality of wafers comprises a shape memory material.

9. The heart valve implant of claim 8, wherein the shape memory material comprises nitinol.

10. The heart valve implant of claim 1, wherein the stacked array of wafers exhibits a tapered shape extending from a proximal end to a distal end.

11. The heart valve implant of claim 1, wherein the stacked array of wafers exhibits a diamond-like shape.

12. The heart valve implant of claim 1, wherein the plurality of wafers have variable cross-sectional diameters.

13. The heart valve implant of claim 12, wherein the cross-sectional shape of the stacked array of wafers includes at least one of round, pear-shaped, elliptical, hour-glass shaped, triangular, or heart shaped.

14. The heart valve implant of claim 1, wherein the plurality of wafers have substantially equal cross-sectional diameters.

15. The heart valve implant of claim 14, wherein the cross-sectional shape of the stacked array of wafers includes at least one of rectangle or square.

16. The heart valve implant of claim 1, wherein each of the plurality of wafers is slidably coupled to the shaft.

17. The heart implant of claim 1, wherein each of the plurality of the wafers is fixedly coupled to the shaft.

18. The heart valve implant of claim 1, further comprising a plurality of coupling members, wherein each of the plurality of coupling members is configured to couple at least one of the wafers to the shaft.

19. The heart valve implant of claim 1, further comprising one or more radiopaque markers.

20. The heart valve implant of claim 19, wherein the one or more radiopaque markers are disposed on the shaft, one or more of the plurality of wafers, or a combination thereof.

21. The heart valve implant of claim 1, wherein the anchor comprises at least one barb, a helical feature, at least one anchor pad, or a combination thereof.

22. The heart valve of claim 1, wherein said implant is a mitral valve implant.

23. A method of delivering a heart valve implant, the method comprising:
implanting the heart valve implant in a patient's heart, the heart valve implant comprising:
a shaft having a first end and a second end;
an anchor coupled to said first end of said shaft, the anchor configured to secure the heart valve implant to heart tissue; and
a plurality of wafers, wherein each of the plurality of wafers is coupled to the second end of the shaft;
at least partially collapsing said plurality of wafers of said heart valve implant;
wherein implanting the heart valve implant comprises:
percutaneously inserting said heart valve implant into the heart;
securing said anchor of said heart valve implant to native coronary tissue of the heart; and
expanding the plurality of wafers to form a stacked array of wafers at least partially within a heart valve to at least partially restrict a flow of blood through a heart valve during systole upon contact with at least a portion of a valve leaflet of the heart valve.

24. The method of claim 23, wherein said step of percutaneously inserting the heart valve implant comprises using a catheter.

25. The method of claim 24, further including inserting the collapsed plurality of wafers into a lumen of the catheter and delivering said heart valve implant to a ventricle of the heart via said catheter.

26. The method of claim 23, further including securing the heart valve implant to any of an interior surface of the heart, an exterior surface of the heart, or a combination thereof.

27. The method of claim 23, wherein one or more of the plurality of wafers comprises a biocompatible polymer.

28. The method of claim 23, wherein one or more of the plurality of wafers comprises a shape memory material.

29. The method of claim 28, wherein the shape memory material comprises nitinol.

30. The method of claim 23, wherein the stacked array of wafers comprises a distal end and an proximal end.

31. The method of claim 23, wherein the heart valve implant further comprises an inflatable balloon that surrounds at least a portion of the plurality of wafers.

32. A method of delivering a heart valve implant, the method comprising:
percutaneously inserting a heart valve implant into a patient's heart, wherein the heart valve implant comprises a shaft extending from a first end to a second end, an anchor coupled to the first end of the shaft, and a plurality of wafers coupled to the shaft in proximity of the second end of the shaft, and wherein said wafers are in an at least partially collapsed state;
securing said anchor of said heart valve implant to native coronary tissue; and
expanding the plurality of wafers to form a stacked array of wafers disposed at least partially within the heart valve so as to at least partially restrict a flow of blood through the heart valve during systole.
